(19) United States
(12) Patent Application Publication
(10) Pub. No.: US 2026/0335640 A1
(43) Pub. Date: Oct. 29, 2026

(54) SYSTEMS AND METHODS FOR STIMULATING ORTHOPEDIC CORRECTIONS (SCOLISTIM)

(71) Applicant: UNIVERSITY OF SOUTH FLORIDA, Tampa, FL (US)

(72) Inventors: Nathan D. SCHILATY, Tampa, FL (US); Jacob CONNOLLY, Tampa, FL (US); Luana NIEWELT, Tampa, FL (US); Avery Kyle ALTAMIRANO, Tampa, FL (US); Stephanie M. APOLO, Tampa, FL (US); Haley DERFLINGER, Tampa, FL (US)

(21) Appl. No.: 19/656,960

(22) Filed: Apr. 23, 2026

Related U.S. Application Data
(60) Provisional application No. 63/793,105, filed on Apr. 23, 2025.

(51) Int. Cl. A61N 1/36; A61N 1/04

(52) U.S. Cl. CPC A61N 1/36003; A61N 1/0452; A61N 1/0456; A61N 1/0484

(57) ABSTRACT

Wearable orthopedic stimulation systems and methods for treating orthopedic conditions affecting a patient's musculoskeletal system are disclosed. In some embodiments, a wearable garment is contemplated which comprises or carries electrodes or an electrode array for muscle stimulation. A therapeutic stimulation plan may be generated and used, which specifies which electrodes are activated and assigns one or more electrical stimulation modalities to activated electrodes based on electrode location relative to target musculature. The stimulation plan may be determined based on clinical analysis, including identification of concave and convex aspects of a spinal curvature. Assignment of electrical muscle stimulation (EMS) and/or transcutaneous electrical nerve stimulation (TENS) is correspondingly determined to promote corrective neuromuscular effects. Usage data or impact/effect data may be transmitted to a companion application for compliance monitoring and updating the stimulation plan.

### CROSS-REFERENCE TO RELATED APPLICATIONS

This application claims priority to U.S. provisional patent application No. 63/793,105, filed on Apr. 23, 2025, the entire contents of which are incorporated herein by reference.

### STATEMENT REGARDING FEDERALLY SPONSORED RESEARCH

N/A

### BACKGROUND

Orthopedic conditions affecting the musculoskeletal system, including spinal deformities, postural disorders, joint misalignments, and chronic musculoskeletal pain syndromes, represent a significant burden on patient populations across a wide range of ages and clinical presentations. Such conditions may include, by way of non-limiting example, scoliosis (including adolescent idiopathic scoliosis (AIS)), kyphosis, lordosis, spondylolisthesis, degenerative disc disease, and various forms of chronic back and joint pain. Taking AIS as one illustrative example, AIS is a spinal condition characterized by abnormal lateral curvature of the spine. The severity of such curvature is conventionally quantified using the Cobb angle, a radiographic measurement derived from an anteroposterior spinal X-ray. To obtain the Cobb angle, the most tilted vertebrae at the upper and lower boundaries of the curve are identified, and lines are drawn along the endplates of those vertebrae. Perpendicular lines are then constructed extending from each endplate line, and the Cobb angle is measured as the angle at which the two perpendicular lines intersect. A Cobb angle measurement exceeding 10 degrees is generally considered diagnostic of scoliosis. AIS is estimated to affect approximately 2-4% of the adolescent population.

Current management approaches for orthopedic conditions such as AIS generally range from clinical observation for mild presentations, rigid bracing or orthotic support for moderate presentations, and surgical intervention for severe cases. For example, in the case of AIS, rigid bracing is widely prescribed for moderate curvatures (Cobb angle measurements of approximately 20-45 degrees), while surgical intervention may be indicated for severe curvatures with Cobb angle measurements in the range of 45-50 degrees. However, across a broad spectrum of orthopedic conditions, conventional orthotic and bracing interventions have been associated with challenges that collectively reduce the therapeutic efficacy of the prescribed regimen. Such challenges include poor patient compliance, psychosocial burden, physical discomfort, and limitations in daily activity.

Electrical stimulation modalities, including transcutaneous electrical nerve stimulation (TENS) and electrical muscle stimulation (EMS), have been explored as potential non-invasive approaches for addressing neuromusculoskeletal aspects of various orthopedic conditions, including pain management, muscle strengthening and re-education, reduction of muscle spasm, improvement of local circulation, and correction of muscular imbalances associated with spinal and postural deformities. For example, in the context of AIS, electrical stimulation has been investigated for its potential to address paraspinal muscle imbalance and associated pain. Similarly, TENS and EMS modalities have been explored in connection with post-operative orthopedic rehabilitation, chronic low back pain, cervical and lumbar postural disorders, and degenerative joint conditions. However, existing approaches have not adequately addressed the integration of targeted, condition-specific electrical stimulation with wearable orthotic support in a manner that is tailored to the individual patient's clinical presentation and compatible with daily activity.

Accordingly, a need exists for wearable, non-invasive devices that integrate therapeutic electrical stimulation with spinal orthotic support in a form factor that supports patient compliance, permits daily activity during therapy, and enables clinician-configurable stimulation parameters tailored to the individual patient's spinal curvature profile.

### SUMMARY

The following presents a simplified summary of the disclosed technology herein in order to provide a basic understanding of some aspects of the present disclosure. This summary is, of course, not a comprehensive or extensive overview of the full content of the present disclosure nor of all perspectives or wording by which the novel, non-obvious, and useful subject matter hereof could be described or presented. It is not meant to specify any particular ‘key,’ ‘necessary,’ or ‘critical’ elements of the disclosed technology nor to delineate the scope of the present disclosure or any claims which might be based hereon. Rather, it is meant to present concise and ‘summary’ examples of how some concepts of the disclosed technology can be described.

In some embodiments, the present disclosure provides a method for treating an orthopedic condition affecting the musculoskeletal system of a patient, comprising: determining a stimulation plan for a wearable orthopedic stimulation device worn on the patient, wherein the stimulation plan specifies activation of a subset of a plurality of electrodes distributed across the wearable device and assigns at least one electrical stimulation modality to each activated electrode based on the location of that electrode relative to target musculature associated with the orthopedic condition; and delivering electrical stimulation to the target musculature in accordance with the stimulation plan.

In some embodiments, determining the stimulation plan comprises receiving clinical imaging data of the patient and, based at least in part on the clinical imaging data, determining which electrodes are to be activated and assigning at least one of transcutaneous electrical nerve stimulation (TENS) or electrical muscle stimulation (EMS) to each activated electrode based on that electrode's location relative to the target musculature.

In some embodiments in which the orthopedic condition is scoliosis, determining the stimulation plan further comprises: identifying, from the clinical imaging data, a concave aspect and a convex aspect of a spinal curvature of the patient; assigning at least one electrode positioned in proximity to the convex aspect to deliver EMS to promote contraction and strengthening of paraspinal musculature on the convex side; and assigning at least one electrode positioned in proximity to the concave aspect to deliver TENS to promote relaxation of paraspinal musculature on the concave side.

In some embodiments, the method further comprises performing a stimulation placement calibration prior to delivering stimulation, comprising: applying a test signal through the plurality of electrodes; measuring impedance at each electrode; determining, based on the measured impedance values, which electrodes are in conductive contact with target musculature; and optionally deriving an approximation of the patient's spinal trajectory based on the pattern of impedance values across the array.

In some embodiments, the method further comprises monitoring patient compliance via a companion application that receives session data over a wireless link, including at least one of: time worn, stimulation modalities delivered, electrode contact status, and stimulation intensity settings. In some embodiments, an inertial measurement unit (IMU) is used to determine whether the wearable device is being worn by the patient.

In some embodiments, the wearable orthopedic stimulation device is configured as a lumbar wrap, a wearable shirt, or a vest providing coverage spanning from upper thoracic to lumbar spinal regions.

In some embodiments, the wearable orthopedic stimulation device is used as an adjuvant therapy alongside a conventional rigid orthopedic brace, wherein the electrical stimulation provides at least one of pain relief and neuromuscular activation to promote patient compliance with the orthopedic brace.

In some embodiments, the present disclosure provides a system for treating an orthopedic condition, comprising: a wearable garment configured to be worn on a patient's torso; a plurality of electrodes distributed across the wearable garment and configured to deliver electrical stimulation to target musculature of the patient; a controller configured to receive a stimulation plan and selectively activate electrodes in accordance with the plan; and a wireless communication module configured to transmit session data to a companion application.

The foregoing is a simplified summary of some aspects of the disclosure and is not intended to be limiting. Additional aspects, features, and advantages will be apparent from the detailed description and drawings set forth below.

### BRIEF DESCRIPTION OF THE DRAWINGS

The foregoing features of embodiments will be more readily understood by reference to the following detailed description, taken with reference to the accompanying drawings, in which:

FIGS. 1A, 1, and 1C illustrate an example wearable orthopedic muscle stimulation device and associated components in accordance with one or more aspects of the present disclosure.

FIG. 1D illustrates an example wearable orthopedic muscle stimulation device having an electrode array configuration in accordance with one or more aspects of the present disclosure.

FIG. 2 is a conceptual schematic demonstrating association between an example clinical assessment and usage of a therapeutic device embodiment, including a diagnostic X-ray image, in accordance with one or more aspects of the present disclosure.

FIG. 3 illustrates an example stimulation garment embodied as a wearable, compressive top with embedded electrodes in accordance with one or more aspects of the present disclosure.

FIG. 4 is a flowchart illustrating an example process for determining and delivering a treatment plan for a patient with an orthopedic condition in accordance with one or more aspects of the present disclosure.

FIG. 5 shows a block diagram illustrating a system for determining an optimal treatment plan and delivering electrical stimulation for an orthopedic condition in accordance with one or more aspects of the present disclosure.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. It should be understood that the processes described below are not limiting of the scope of this disclosure, can be combined in various configurations (in whole or in part), and may be adapted to replace, complement, and/or fit with attributes and needs of different clinical, therapeutic, and patient-care contexts.

### Example Wearable Stimulation Device and Component Layout

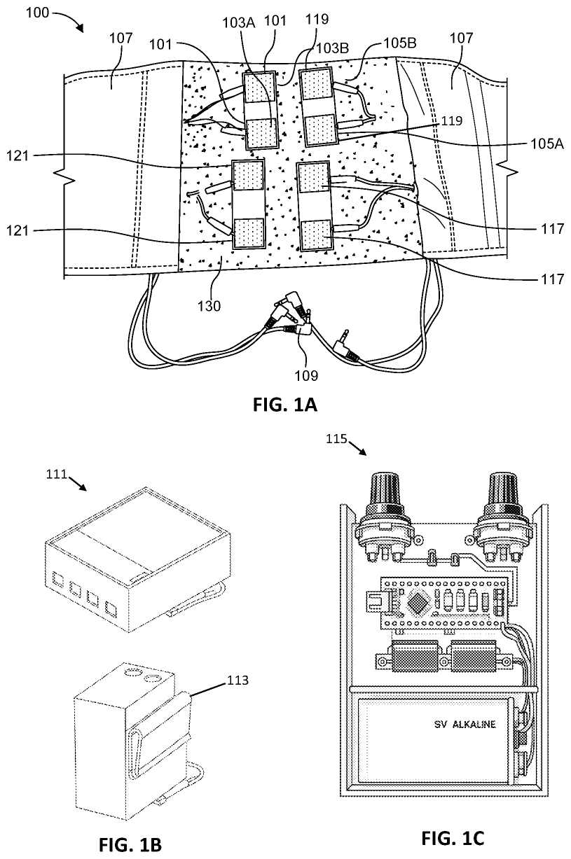

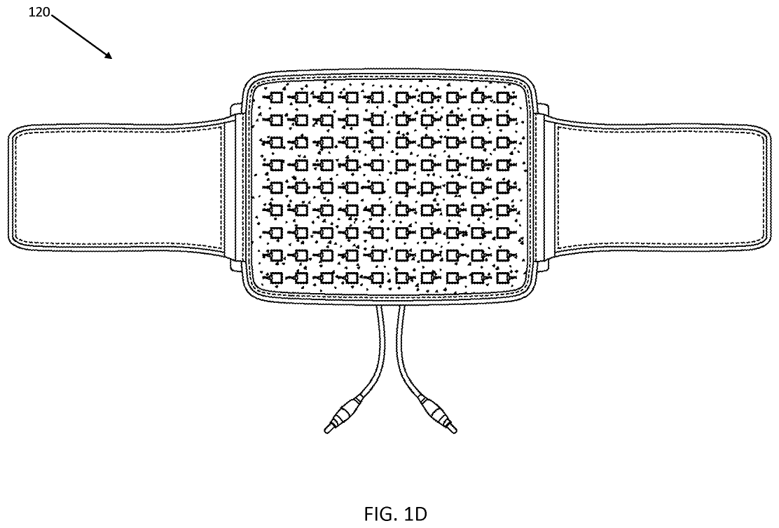

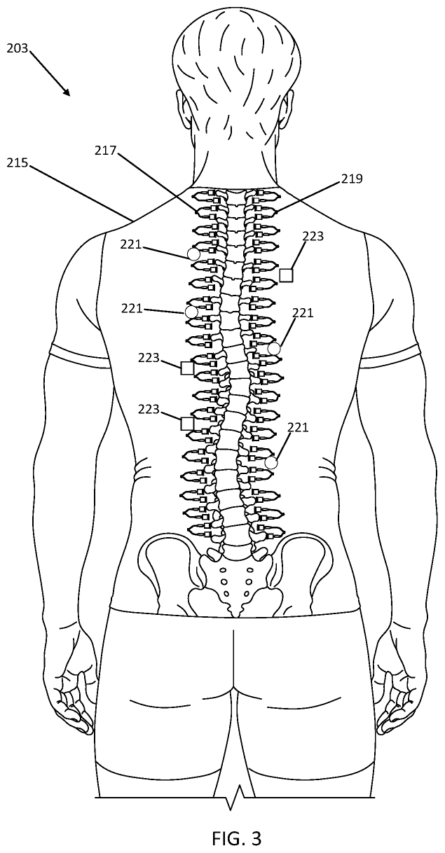

FIG. 1A illustrates an example wearable orthopedic muscle stimulation device 100 in accordance with one or more aspects of the present disclosure. In some embodiments, the device 100 may be a wearable wrap designed for individuals presenting with orthopedic conditions affecting the musculoskeletal system, including but not limited to spinal deformities (such as scoliosis, kyphosis, or lordosis), postural disorders, joint misalignments, chronic musculoskeletal pain syndromes, and conditions requiring post-surgical orthopedic rehabilitation. In some embodiments, the device 100 may take the form of a lumbar wrap or band, configured to be worn about the patient's lower torso and targeting the lumbar and thoracolumbar musculature. In other embodiments, the device 100 may take the form of a full wearable shirt (see FIG. 3), providing electrode coverage across a broader extent of the patient's musculature. For example, in the case of scoliosis, where curvature is commonly thoracolumbar in character, a shirt configuration may provide electrode coverage spanning from the upper thoracic spine through the lumbar region, encompassing the full extent of the curvature. In further embodiments, the device 100 may take the form of a vest, a brace, a compression garment, a shoulder or extremity wrap, or any other wearable article configured to position the electrode components described herein in proximity to the patient's target musculature associated with the diagnosed orthopedic condition. In some embodiments, the device 100 may be worn under the patient's ordinary clothing, such as under a T-shirt, supporting patient comfort and discretion throughout daily activities. In some embodiments, the device 100 may be used as a standalone primary intervention. In other embodiments, the device 100 may be used as an adjuvant therapy alongside a conventional rigid orthopedic brace or orthotic device, providing pain relief and muscle stimulation that promotes greater patient compliance with the prescribed orthotic regimen. In further embodiments, the device 100 may be configured for prophylactic use in patients presenting with early-stage neuromusculoskeletal markers of an orthopedic condition (for example, early-stage scoliosis), intervening before the condition has progressed to the threshold conventionally associated with rigid bracing or surgical intervention, and, in the case of spinal deformities, before bone remodeling results in a permanent structural deformity.

In some embodiments, the device 100 integrates four channels of adjustable electrically conductive electrodes for example, silver/silver chloride fabric electrode patches with two channels positioned on each side of the spinal column to provide bilateral paraspinal coverage. In other embodiments, the device 100 may integrate two or more channels of adjustable conductive electrodes, with the number of channels selected based on the patient's anatomy, the extent of the spinal curvature to be addressed, and the desired stimulation coverage area. The device 100 connects to a Transcutaneous Electrical Nerve Stimulation (TENS) unit and an Electrical Muscle Stimulation (EMS) unit, aimed to alleviate pain and strengthen targeted musculature associated with the patient's diagnosed condition. It should be understood that the processes, components, and configurations described with respect to FIG. 1 are illustrative of certain embodiments, and that the device 100 may be adapted, combined, or modified in accordance with the various further embodiments described herein without departing from the scope of the disclosure.

In some embodiments, the device 100 may be configured to receive and retain electrode pads at any of a plurality of locations across the body of the wrap, enabling clinician-specified placement of electrode pads throughout the device to target the patient's musculature as clinically indicated. For example, as shown in FIG. 1A, electrode pads may be placed at a first electrode pair position 101, a second electrode pair position 117, a third electrode pair position 119, and a fourth electrode pair position 121, though it should be understood that these positions are illustrative and that electrode pads may be positioned at other locations across the device as determined by the treating clinician. Each electrode pair position corresponds to a location at which one or more electrode pads are retained in contact with the patient's skin overlying the target musculature.

In some embodiments, the electrode pads may comprise adjustable conductive mounting patches positionable at clinician-specified locations across the device 100. In some embodiments, the device 100 may include physical alignment markings disposed on the surface of the wrap, such as an indicator configured to align with the patient's navel and a marking configured to run parallel to the beltline, so that the patient is able to position the device 100 consistently and reproducibly from session to session without clinical supervision. In further embodiments, the device 100 may include tactile features, such as raised ridges or contoured sections, that guide the patient to orient the wrap relative to anatomical landmarks without requiring visual inspection.

In some embodiments, the device 100 may be configured to support an initial calibration protocol upon application, such as a sequence of patient movements for example, bending forward and standing upright to confirm adequate electrode-to-skin contact across the electrode array before commencing stimulation. In other embodiments, a small electrical resistance measurement may be applied across the electrode array to map which electrode pads are in contact with paraspinal muscle tissue and which overlie bony prominence, enabling inference of the spinal trajectory and automatic assignment of electrode pairs to the appropriate TENS or EMS stimulation zone. In further embodiments, recalibration of the electrode placement may occur on a periodic basis, for example, weekly or bimonthly, to account for changes in the patient's spinal alignment over the course of treatment. Periodic recalibration is warranted in part because, as the patient's spinal curvature responds to treatment and alignment progressively improves, the spatial relationship between the electrode array and the target paraspinal musculature shifts electrodes previously assigned to the concave-side TENS zone or the convex-side EMS zone may no longer correspond to the same anatomical positions as the patient's spinal trajectory changes. Accordingly, periodic recalibration enables reassignment of electrode contacts to updated concave-side and convex-side stimulation zones to maintain therapeutic accuracy as treatment progresses. In some embodiments, the companion application continuously monitors session data and postural metrics to identify changes in electrode impedance patterns that may indicate clinically meaningful shifts in spinal alignment, and generates a recalibration prompt to the patient and/or clinician when such changes are detected.

In some embodiments, electrode contact verification is alternatively performed via a sequential trial-and-error detection routine, in which the circuit device 115 applies a low-level test signal to individual electrode pairs sequentially and measures the resulting current or impedance to determine whether adequate conductive contact has been established at each electrode location. Electrode pairs that fail to achieve a minimum conductance threshold during the sequential scan are identified and flagged for positional correction prior to commencement of stimulation delivery. In some embodiments, this trial-and-error detection routine is performed automatically at the start of each treatment session and does not require any predetermined electrode placement map, making it suitable for use in embodiments where the garment is donned by the patient independently without prior clinician-guided placement.

In some embodiments, the device 100 may include a strap or compressive wrap portion 107 comprising a structural fabric band configured to secure the wrap about the patient's torso or other target body region and to maintain consistent contact pressure between the electrode pads and the patient's skin throughout a treatment session. In some embodiments, the compressive wrap portion 107 may comprise a double-layer configuration, wherein an outer wrap layer is applied first and an inner elastic band further stretches and compresses the material against the patient's body, with the inner band secured via a hook-and-loop closure to ensure adherence through double-layer elasticity, maintaining electrode-to-skin contact even across irregular body contours that may otherwise produce a tenting effect in a single-layer fabric wrap. Such irregular body contours may include, for example, the convex geometry of a scoliotic back or other asymmetric musculoskeletal presentation. In other embodiments, the compressive wrap portion 107 may be fabricated from an elastic or stretch fabric, such as neoprene, spandex, or a dry-fit synthetic material, configured to conform to the patient's body while providing sufficient inward compression to maintain electrode-to-skin contact across a range of body morphologies. In further embodiments, the compressive wrap portion 107 may incorporate conductive threads or fibers woven into selected zones, enabling the strap itself to function as an extended electrode contact surface in addition to, or in place of, the discrete electrode pads. In some embodiments, the compressive wrap portion 107 may incorporate one or more hook-and-loop fasteners, snap closures, buckle systems, or other adjustable closure mechanisms to accommodate a range of patient body circumferences and to allow the device 100 to be self-applied and adjusted by the patient without clinical assistance.

In some embodiments, the device 100 may include silver fabric electrode pads arranged in pairs and distributed across the pair positions of the device 100. For example, as shown in FIG. 1A, a first electrode pair at electrode pair position 101 comprises a first electrode pad 103A and a second electrode pad 103B, and a second electrode pair at electrode pair position 119 comprises a first electrode pad 105A and a second electrode pad 105B, with additional electrode pairs located at electrode pair positions 117 and 121, each pair corresponding to a respective stimulation channel of the device 100 and positioned at a clinician-specified location along the patient's target musculature. In some embodiments, the device 100 may include additional electrode pairs beyond those shown in FIG. 1A, such that the device 100 integrates multiple channels of adjustable silver fabric electrodes positioned at clinician-specified locations along the patient's target musculature.

In some embodiments, each electrode pair may comprise a source electrode and a sink electrode functioning as the two poles of a stimulation circuit for the corresponding channel, positioned such that the electrical stimulation signal travels along the target muscle fiber in a direction consistent with the desired therapeutic effect for example, vertically along the paraspinal muscle fibers in the case of spinal deformities. In some embodiments, each electrode pad may be fabricated from silver chloride fabric material, such that electrical conductivity to the skin contact area causes the pad to function as an electrode, with the silver chloride fabric relying on skin contact and perspiration over time to achieve sufficient conductivity for reliable stimulation delivery. In other embodiments, the electrode pads may be supplemented with a conductive gel at the skin-contact surface to enhance conductivity, particularly for patients with lower perspiration or greater subdermal tissue thickness. In further embodiments, the electrode pads may be integrated directly into the fabric of the wrap 100 as fixed electrodes in a predetermined array (see, e.g., FIG. 1D), rather than being individually positionable patches, with selective activation of array elements determined based on clinical imaging data or real-time resistance mapping.

FIG. 1D illustrates an example wearable orthopedic muscle stimulation device 120 in accordance with one or more aspects of the present disclosure. The device 120 may be similar to the device 100 described with respect to FIGS. 1A-1C, except that the device 120 includes an electrode array comprising a plurality of TENS electrodes and EMS electrodes arranged in a grid or matrix configuration integrated into the body of the wrap, rather than employing individually positionable discrete electrode pads. In some embodiments, the electrode array of the device 120 may comprise individually addressable electrode elements, each of which may be selectively assigned by the treating clinician to deliver TENS stimulation, EMS stimulation, or no stimulation, based on the patient's diagnosed orthopedic condition and the corresponding stimulation plan. In some embodiments, the treating clinician may configure the electrode array of the device 120 via the circuit device 115 or a companion application to modify the assignment of individual electrodes between TENS and EMS modalities, adjust the spatial distribution of active electrodes across the array, and update the stimulation parameters associated with each electrode or group of electrodes, enabling the clinician to tailor the electrode activation pattern to the patient's evolving clinical presentation without requiring physical repositioning of discrete electrode pads. In some embodiments, the electrode array of the device 120 may be reconfigured at periodic clinical visits for example, upon review of updated diagnostic imaging to reflect changes in the patient's spinal curvature profile, with the treating clinician reassigning electrodes from EMS to TENS or vice versa as the muscular imbalance associated with the orthopedic condition is progressively corrected. In some embodiments, the circuit device 115 of the device 120 may be embodied as a compact, reduced-footprint unit having a generally circular form factor, configured to minimize the physical profile of the electronics module against the patient's body and to facilitate unobtrusive integration with the electrode array of the device 120 during daily wear.

In some embodiments, the electrode pads may comprise hydrogel electrode pads, wherein a pre-gelled adhesive hydrogel layer is disposed at the skin-contact surface of each electrode pad, providing consistent and uniform conductivity independent of the patient's perspiration level and enabling reliable stimulation delivery from the onset of a treatment session without requiring a conditioning period.

In other embodiments, the electrode pads may comprise carbon-filled elastomer electrode pads fabricated from a conductive rubber or silicone material impregnated with carbon particles, configured to provide a flexible, reusable, and conformable electrode surface that adapts to the contours of the patient's body, including irregular or asymmetric body surfaces such as those associated with scoliotic deformity.

In further embodiments, the electrode pads may comprise dry electrodes that do not require gel, adhesive, or moisture at the skin-contact interface, and may be fabricated from conductive polymers, gold-plated or silver-plated metallic contacts, stainless steel discs, or micro-needle arrays configured to achieve low-impedance electrical coupling to the skin without the application of a conductive medium, thereby reducing skin irritation and preparation time associated with gel-based electrode configurations.

In some embodiments, the electrode pads may comprise textile electrodes woven or knitted from conductive yarns such as silver-coated nylon, stainless steel fibers, or carbon-fiber blended yarns directly into the fabric substrate of the device 100, providing an electrode surface that is mechanically integrated with the garment and that conforms to the patient's body through the natural drape and stretch of the textile, without requiring a discrete electrode pad to be separately attached to the garment.

In further embodiments, the electrode pads may comprise capacitive coupling electrodes configured to deliver electrical stimulation through a thin dielectric layer interposed between the conductive electrode surface and the patient's skin, such that stimulation is delivered via capacitive displacement current rather than direct galvanic contact, potentially reducing the risk of skin irritation, electrochemical burns, or allergic reaction associated with prolonged direct-contact electrode use.

In some embodiments, the electrode pads may comprise multi-layer or composite electrode pads incorporating two or more functional layers for example, a conductive layer providing the electrical interface, a moisture-management layer configured to wick perspiration away from the skin-contact surface or to distribute a conductive medium uniformly across the electrode area, an adhesion layer configured to maintain the electrode pad in stable contact with the patient's skin, and a biocompatible skin-interface layer configured to minimize irritation, allergic response, or dermatological adverse effects during extended wear with the composition and arrangement of layers selected based on the intended duration of wear, the patient's skin sensitivity, and the stimulation modality to be delivered.

In some embodiments, the electrode pads may be provided in a plurality of sizes and shapes including rectangular, circular, oval, annular, and anatomically contoured configurations selectable by the treating clinician based on the dimensions of the target muscle group, the anatomical region to be stimulated, and the patient's body morphology, such that the electrode contact area may be matched to the clinical requirements of the individual patient's treatment plan. In other embodiments, the electrode pads may be configured as replaceable or disposable components, removably attached to the device 100 via snap connectors, hook-and-loop patches, magnetic couplings, or other releasable attachment mechanisms, enabling the patient or clinician to replace individual electrode pads (for example, upon degradation of the conductive surface, depletion of a hydrogel layer, or loss of adhesion) without replacing the device 100 as a whole, thereby supporting long-term use and reducing the cost of ongoing treatment.

In some embodiments, as shown in FIG. 1A, the device 100 may include an electrode attachment substrate 130 disposed on the inner surface of the compressive wrap portion 107 at each electrode pair position and configured to releasably secure the electrode pads to the body of the device 100 at clinician-specified locations. In some embodiments, the electrode attachment substrate 130 may comprise a hook-and-loop fastener material, wherein a loop field is affixed to the inner surface of the compressive wrap portion 107 and a corresponding hook patch is affixed to the rear surface of each electrode pad, enabling the electrode pad to be pressed onto and peeled away from the loop field to permit repositioning of the electrode pad along the device 100 as clinically indicated. In other embodiments, the electrode attachment substrate 130 may comprise a medical-grade adhesive layer disposed on the inner surface of the compressive wrap portion 107, configured to provide a tacky, repositionable bond between the electrode pad and the garment surface that maintains the electrode pad in position during a treatment session while permitting removal and repositioning without damaging the garment or the electrode pad. In further embodiments, the electrode attachment substrate 130 may comprise a magnetic coupling, wherein one or more magnets or ferromagnetic elements are embedded in the compressive wrap portion 107 at each electrode pair position and corresponding magnets or ferromagnetic elements are embedded in or affixed to the electrode pad, such that the electrode pad is held in position by magnetic attraction and may be repositioned by overcoming the magnetic holding force. In some embodiments, the electrode attachment substrate 130 may comprise a snap fastener system, wherein male snap elements are disposed on the inner surface of the compressive wrap portion 107 and female snap receptacles are disposed on the rear surface of each electrode pad, or vice versa, providing a positive mechanical engagement that resists displacement during patient movement. In some embodiments, the electrode attachment substrate 130 may comprise a conductive adhesive or conductive hook-and-loop material that simultaneously secures the electrode pad to the garment and provides an electrical pathway between the electrode pad and a conductive trace routed through the compressive wrap portion 107, thereby combining mechanical attachment and electrical interconnection in a single substrate layer. In some embodiments, the electrode attachment substrate 130 may comprise a silicone friction pad or elastomeric grip layer configured to retain the electrode pad in position through frictional engagement with the rear surface of the electrode pad, without requiring adhesive, mechanical fasteners, or magnetic elements, and permitting repositioning by sliding the electrode pad along the grip surface.

In some embodiments, electrode pairs of the device 100 may be configured to deliver differentiated stimulation to distinct muscle groups or anatomical zones based on the patient's diagnosed orthopedic condition. For example, in the case of scoliosis, electrode pairs positioned on the concave side of the patient's spinal curvature may be configured to deliver TENS stimulation at parameters configured to promote muscle relaxation in the tight paraspinal muscles on that side, while electrode pairs positioned on the convex side may be configured to deliver EMS stimulation at parameters configured to promote active muscle contraction and strengthening in the comparatively lax paraspinal muscles on that side.

In some embodiments, the zonal assignment of TENS and EMS stimulation may be configured to address the particular muscular imbalances, pain patterns, or rehabilitation targets associated with the patient's condition for example, targeting weakened stabilizer muscles in post-surgical rehabilitation, or addressing bilateral muscle spasm in chronic low back pain.

In some embodiments, the TENS stimulation parameters may include an intensity in the range of approximately 5V to 80V, a pulse width in the range of approximately 20 microseconds to 400 microseconds, and a frequency in the range of approximately 1 Hz to 150 Hz. In some embodiments, the TENS stimulation parameters may include an intensity in the range of approximately 10V to 50V, a pulse width in the range of approximately 50 microseconds to 100 microseconds, and a frequency in the range of approximately 50 Hz to 120 Hz. In one example, the TENS stimulation parameters may include an intensity of approximately 18.2V, a pulse width of approximately 72 microseconds, and a frequency of approximately 93 Hz. In some embodiments, the EMS stimulation parameters may include an intensity in the range of approximately 5V to 100V, a pulse width in the range of approximately 100 microseconds to 1000 microseconds, and a frequency in the range of approximately 1 Hz to 100 Hz. In some embodiments, the EMS stimulation parameters may include an intensity in the range of approximately bV to 50V, a pulse width in the range of approximately 200 microseconds to 500 microseconds, and a frequency in the range of approximately 30 Hz to 80 Hz. In one example, the EMS stimulation parameters may include an intensity of approximately 18.4V, a pulse width of approximately 335 microseconds, and a frequency of approximately 63 Hz. In some embodiments, the TENS and EMS stimulation parameters may be adjustable by the treating clinician via the circuit device 115 and its associated interface to titrate stimulation to the individual patient's morphology, pain threshold, and treatment stage, with such parameters adjustable over the course of treatment to reflect the patient's current condition and therapeutic progression. In further embodiments, as the patient's musculoskeletal condition improves, the assignment of electrode pairs to TENS or EMS stimulation may be updated to reflect the patient's progress, with stimulation patterns adjusted accordingly. For example, stimulation patterns may become progressively more symmetric as spinal curvature correction is achieved in scoliosis patients.

In some embodiments, the device 100 may include electrode lead wires 109 configured to electrically couple the electrode pads 103A, 103B, 105A, and 105B to the circuit device 115, transmitting the TENS and EMS stimulation signals generated by the circuit device 115 to the respective electrode pairs in accordance with the programmed treatment parameters. In some embodiments, to activate therapy, the patient may put on the wrap 100 and connect the lead wires 109 to the circuit device 115, enabling muscle stimulation without requiring clinical assistance. In other embodiments, the lead wires 109 may be routed along or within the body of the compressive wrap portion 107, minimizing interference with the patient's freedom of movement and reducing the risk of accidental disconnection during daily activities. In further embodiments, the lead wires 109 may include quick-disconnect connectors at their termination points, allowing the electrode pads to be detached and repositioned along the device 100 without replacing the entire lead wire assembly. In some embodiments, the lead wires 109 may be replaced by conductive traces printed or woven directly into the fabric of the wrap 100, eliminating discrete external wiring and reducing the bulk and complexity of the device assembly. In other embodiments, the lead wire connectors may incorporate locking or keyed features configured to prevent inadvertent disconnection during use, particularly in applications involving pediatric patients.

In some embodiments, the device 100 may include a circuit device housing 111 (see FIG. 1B) configured to contain the printed circuit board (PCB) and associated electronics that generate and deliver the TENS and EMS stimulation signals to the electrode pads via the lead wires 109. In some embodiments, the housing 111 may be fabricated by additive manufacturing from a rigid polymer material configured to protect the enclosed electronics from mechanical damage, moisture, and deformation during active wear. In other embodiments, the housing 111 may be fabricated from injection-molded thermoplastic, die-cast metal, or a semi-rigid material adapted to conform partially to the patient's body, reducing bulk and improving wearability. In further embodiments, the housing 111 may incorporate environmental sealing, such as an Ingress Protection (IP)-rated gasket or conformal coating on the enclosed PCB, to resist perspiration and moisture ingress during extended therapy sessions. In some embodiments, the housing 111 may be integrated directly into the body of the wrap 100, for example by being bonded into a pocket or compartment within the fabric of the compressive wrap portion 107, rather than being carried as a discrete external unit. In other embodiments, the housing 111 may include a transparent or translucent window or indicator region allowing the patient to view the operational status of the circuit device 115 such as a power-on or active stimulation indicator without opening the housing.

In some embodiments, the device 100 may include an attachment clip 113 disposed on the exterior surface of the circuit device housing 111 and configured to secure the housing 111 to the patient's clothing, belt, or to the compressive wrap portion 107, maintaining the circuit device 115 (see FIG. 1C) in a fixed and accessible position relative to the patient's body during a treatment session. In some embodiments, the attachment clip 113 may comprise a spring-loaded belt clip, a hook-and-loop patch, a magnetic closure, a snap fastener, or any other suitable attachment mechanism configured to hold the housing 111 against the patient's body or garment without requiring tools or clinical assistance for attachment or removal. In other embodiments, the attachment clip 113 may be adjustable or repositionable, allowing the patient or clinician to select a carrying position such as on the hip, lower back, or abdomen that provides comfort and accessibility for the patient's particular morphology and treatment configuration. In further embodiments, the attachment clip 113 may incorporate a rotatable or pivoting joint allowing the housing 111 to articulate relative to the clip base, accommodating movement of the patient's body without placing torque on the attachment point. In some embodiments, the attachment clip 113 may be detachable from the housing 111, permitting the housing 111 to be placed on a flat surface during charging or programming operations, with the clip reattached prior to wear.

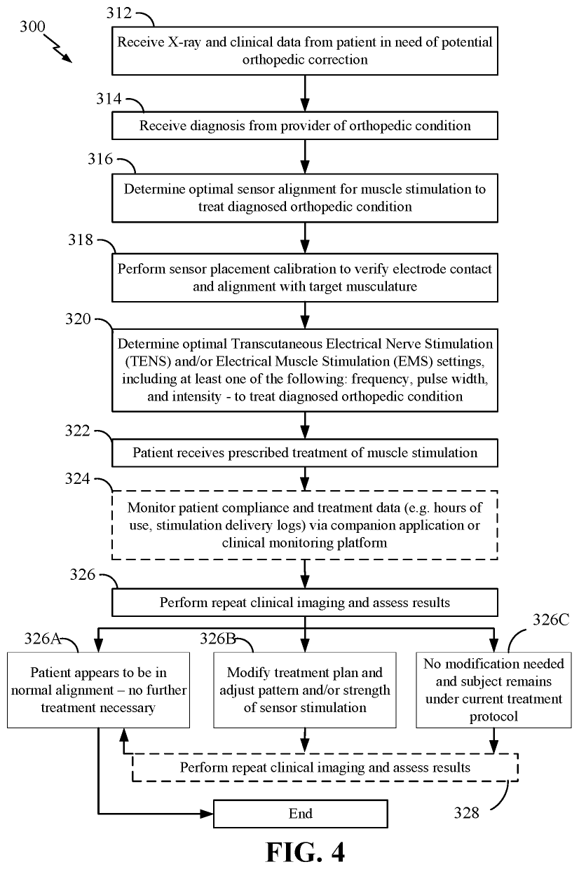

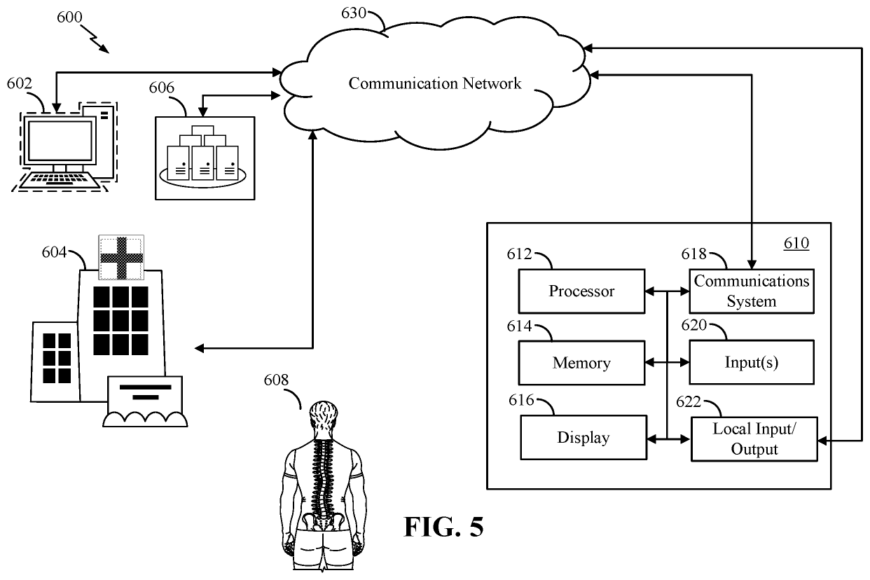

In some embodiments, the device 100 may include a circuit device 115 comprising the PCB and associated electronics housed within the enclosure 111 and configured to generate and control delivery of the TENS and EMS electrical stimulation signals to the electrode pads 103A, 103B, 105A, and 105B via the lead wires 109. In some embodiments, the circuit device 115 may include intensity adjustment controls such as potentiometers permitting adjustment of stimulation output for the respective TENS and EMS channels, as well as a battery and a USB port facilitating programming and communication with the associated interface. In other embodiments, intensity adjustment may be performed digitally via a software interface, enabling finer and more reproducible parameter control and eliminating the need for physical adjustment mechanisms on the housing 111. In some embodiments, the circuit device 115 may be configured via a graphical user interface (GUI) through which the treating clinician sets the TENS and EMS frequency and pulse width parameters to desired values and transmits those parameters to the device 100 prior to or during a treatment session. In other embodiments, the circuit device 115 may incorporate a wireless communication module such as a Bluetooth, Bluetooth Low Energy, Wi-Fi, or other suitable transceiver (see, e.g., communications system 618 of FIG. 5) enabling the circuit device 115 to communicate in real time with a companion mobile application or clinical monitoring platform via communication network 630 (FIG. 5), through which a clinician may track patient compliance, monitor the number of hours per day the patient is wearing the device 100, review the time of day during which the device is active, and receive alerts when usage patterns fall outside of expected parameters, as further described with respect to step 324 of method 300 (FIG. 4). In further embodiments, the device 100 may include one or more inertial measurement units (IMUs) integrated into the body of the wearable garment, each IMU communicatively coupled to the circuit device 115 via a wired connection routed through the garment or a short-range wireless link, and configured to detect the orientation and movement of the device 100, distinguishing, for example, between the device being worn on the patient's body versus resting on a flat surface, as part of a compliance monitoring algorithm executed by the circuit device 115 to determine whether the device is actually being worn during a given period. In some embodiments, the circuit device 115 may monitor electrode resistance values in real time and generate a patient-facing alert if resistance exceeds a threshold indicative of inadequate skin contact, prompting the patient to reposition the device 100 before commencing or continuing a treatment session. In further embodiments, the circuit device 115 may be configured to store multiple patient profiles, allowing the device 100 to be used across more than one patient for example, in a clinical setting with session data logged separately for each profile and transmitted to the respective patient record upon session completion via the communication network 630 (see FIG. 5).

In some embodiments, the compliance monitoring system is further configured to detect potential compliance falsification events. The circuit device 115 measures impedance or resistance at the electrode-skin interface during each recorded wear session and compares the measured values against expected ranges for actual biological tissue contact. Impedance or resistance values inconsistent with tissue contact for example, values indicative of a short-circuit condition or metallic bypass (e.g., a conductive object placed between electrode contacts to simulate skin contact without actual device wear) are flagged as potential falsification events and reported to the clinician through the clinical platform interface. In some embodiments, the compliance monitoring system further analyzes the time-of-day distribution of recorded wear sessions. Wear patterns that are inconsistent with the patient's prescribed treatment schedule for example, sessions recorded exclusively during typical sleeping hours are flagged as potential compliance anomalies for clinician review via the companion application or clinical dashboard.

In some embodiments, the circuit device 115 is configured to automatically titrate the intensity of electrical stimulation delivered to each activated electrode. The automated titration routine incrementally increases stimulation output from a defined baseline level while monitoring one or more feedback signals such as electrode impedance, skin resistance changes, or patient-reported discomfort received via the companion application interface and reduces stimulation intensity upon detection of a threshold discomfort indicator or upon reaching a pre-defined maximum output level. The resulting titrated intensity value is logged by the circuit device 115 and used as the starting point for subsequent treatment sessions, enabling reproducible stimulation delivery across sessions that is independent of subjective patient self-report. In some embodiments, the automated titration routine accounts for inter-patient variability in tissue impedance for example, patients with greater subdermal adipose tissue may require higher stimulation drive levels to achieve equivalent therapeutic effect by incorporating a tissue-impedance correction factor derived from the electrode impedance measurements.

In some embodiments, the one or more IMUs described herein may each comprise one or more accelerometers and one or more gyroscopes configured to measure the orientation, angular position, and movement of the portion of the patient's body to which the IMU is affixed, relative to the gravitational vector. In some embodiments, each IMU may be mounted to the wearable garment via a hook-and-loop attachment, a snap connector, or a fabric pocket, enabling the IMU to be repositioned along the garment as clinically indicated (for example, at the apex of a scoliotic curve, at the cervicothoracic junction, or at the lumbosacral region) to capture orientation data most relevant to the patient's diagnosed condition. Because each IMU measures orientation based on inertial and gravitational reference frames, the IMU output is not affected by the electrical stimulation signals delivered by the TENS and EMS channels of the circuit device 115, enabling concurrent operation of the IMU and the stimulation subsystem without signal interference.

In some embodiments, the circuit device 115 may be configured to receive orientation data from the one or more IMUs in real time and to compare the received orientation data against a reference posture profile established during an initial calibration session (for example, a neutral standing posture recorded at the time of device fitting). When the circuit device 115 detects that the patient's postural orientation has deviated from the reference posture profile by more than a clinician-configurable threshold (for example, a lateral tilt exceeding a prescribed angular deviation in the coronal plane indicative of scoliotic postural asymmetry, or a forward inclination exceeding a prescribed angular deviation in the sagittal plane indicative of kyphotic slouching or cervicothoracic postural deterioration), the circuit device 115 may automatically initiate or increase the intensity of EMS stimulation delivered to the electrode pairs associated with the corrective musculature, thereby prompting the patient to return to the target postural alignment through neuromuscular activation. For example, in a patient presenting with scoliosis, the circuit device 115 may detect a lateral tilt toward the concave side of the curvature and responsively increase EMS intensity to the convex-side paraspinal musculature to promote active postural correction. In a patient presenting with cervicothoracic postural dysfunction, the circuit device 115 may detect forward shoulder posture or excessive thoracic flexion and responsively increase EMS intensity to the posterior shoulder girdle and thoracic extensor musculature to promote an upright posture. In some embodiments, the corrective stimulation response may be graduated, with the circuit device 115 applying progressively increasing stimulation intensity as the magnitude or duration of the postural deviation increases, and reducing stimulation intensity as the patient returns toward the target alignment. In some embodiments, the circuit device 115 may additionally or alternatively generate a patient-facing alert, such as a vibrotactile cue, an audible tone, or a notification transmitted to the companion application, upon detection of a postural deviation, enabling the patient to self-correct before corrective stimulation is applied.

In some embodiments, the circuit device 115 may be configured to log IMU orientation data over the course of each treatment session and across multiple treatment sessions, enabling longitudinal tracking of the patient's postural alignment over time. For example, the circuit device 115 or the companion application may record the patient's baseline angular orientation, such as the degree of lateral tilt in the coronal plane for a scoliosis patient, at the commencement of the treatment regimen, and may compare subsequent session-by-session orientation data against the baseline to quantify postural improvement or deterioration. In some embodiments, the longitudinal IMU data may be transmitted to the clinical database 606 or to the computing device 610 and presented to the treating clinician via the companion application or clinical monitoring platform, providing an objective, non-radiographic measure of the patient's postural response to treatment that supplements periodic clinical imaging assessments. In some embodiments, the treating clinician may use the longitudinal IMU data to adjust the stimulation plan (for example, modifying electrode activation patterns, stimulation modalities, or postural deviation thresholds) based on the patient's measured postural trajectory, thereby enabling data-driven refinement of the treatment protocol between clinical imaging intervals.

### Example Clinical Assessment and Device Configuration

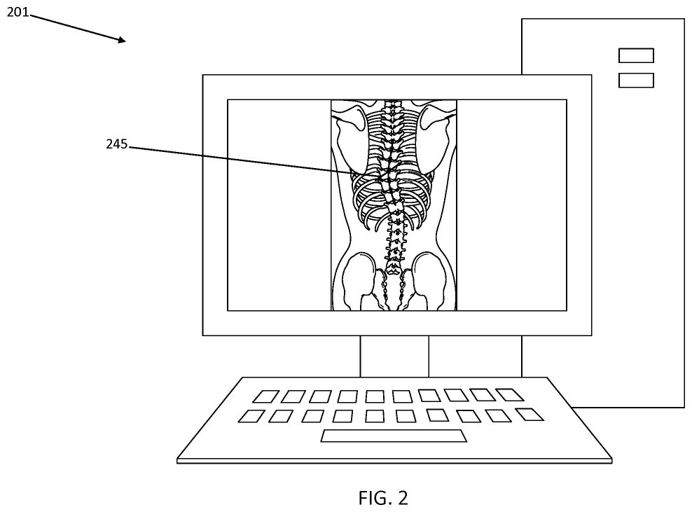

FIG. 2 illustrates an example clinical assessment and device application scenario in accordance with one or more aspects of the present disclosure. As shown in FIG. 2, a diagnostic image 201, such as an X-ray image, of a subject exhibiting scoliosis is depicted alongside the subject wearing a stimulation garment 203 embodying the device 100 described herein. In some embodiments, a treating clinician, such as an orthopedic surgeon, physiatrist, or physical therapist, may review the diagnostic image 201 to assess the type, severity, and spatial extent of the patient's spinal curvature and, based on that assessment, determine which electrodes of the stimulation garment 203 should be activated and what stimulation modality each activated electrode should deliver. In some embodiments, the treating clinician may assess one or more Cobb angles from the diagnostic image 201 or other clinical imaging to characterize the degree and location of the spinal curvature, and may use the assessed Cobb angle measurements to determine which specific electrodes of the stimulation garment 203 should be activated and the stimulation modality to be delivered at each activated electrode. In some embodiments, the treating clinician may use the curvature trajectory visible in the diagnostic image 201 to identify the concave and convex aspects of the curve, to determine the spinal levels at which the curvature originates and terminates, and to trace the trajectory of the affected paraspinal musculature, in particular the erector spinae muscles, along the full extent of the curve. In some embodiments, the diagnostic image 201 may reveal an S-shaped (double) curvature, in which the spine exhibits a primary curve in one direction and a compensatory curve in the opposite direction, with each curve having its own Cobb angle measurement.

In some embodiments, based on the clinical assessment of the diagnostic image 201, the treating clinician may prescribe a stimulation plan specifying which electrodes of the stimulation garment 203 are to be activated, the stimulation modality (TENS or EMS) to be delivered at each activated electrode, and the stimulation parameters (including frequency, pulse width, and intensity) for each channel. In some embodiments, the stimulation plan may direct EMS stimulation to electrodes overlying the convex side of each curve to promote contraction and strengthening of the comparatively lax paraspinal musculature, and may direct TENS stimulation to electrodes overlying the concave side to promote relaxation of the characteristically tight musculature, as described herein with respect to an electrode pair position and circuit device 115. In some embodiments, the treating clinician may further use the diagnostic image 201 to ensure that the prescribed electrode activation pattern targets the erector spinae muscles along the trajectory of the curvature, rather than adjacent non-target musculature such as the latissimus dorsi, so that the delivered stimulation produces the intended therapeutic effect on the muscles most directly associated with the spinal deformity.

As further shown in FIG. 2, the subject is depicted wearing the stimulation garment 203, which in the illustrated embodiment takes the form of a wearable shirt or vest configured to position an electrode array of the device 100 in proximity to the paraspinal musculature along the full extent of the subject's spinal curvature. In some embodiments, the stimulation garment 203 may extend from the upper thoracic spine through the lumbar region, providing electrode coverage across spinal levels T1 through L5 so as to encompass the full thoracolumbar extent of the curvature identified in the diagnostic image 201. In some embodiments, the treating clinician may correlate the curvature trajectory identified in the diagnostic image 201 with the electrode positions of the stimulation garment 203 to configure a stimulation map for the device 100, selecting which individual electrodes or electrode pairs within the array are to be activated and assigning each activated electrode to the appropriate stimulation modality, for example, assigning electrode pairs along the convex aspect of the curve to EMS stimulation and electrode pairs along the concave aspect to TENS stimulation. In some embodiments, the stimulation garment 203 may incorporate an array of individually addressable electrodes, enabling the circuit device 115 to selectively activate subsets of electrodes along the trajectory of the target musculature without requiring physical repositioning of discrete electrode pads, such that the treating clinician's stimulation map derived from the diagnostic image 201 may be implemented electronically by the circuit device 115 without manual electrode placement. In some embodiments, the treating clinician may configure the stimulation map such that multiple source-sink electrode pairs are activated along the trajectory of the erector spinae muscles on the convex side of the curvature to deliver EMS stimulation, while fewer electrode pairs are activated on the concave side to deliver TENS stimulation, reflecting the differential therapeutic requirements of the two sides of the curve. In some embodiments, as the patient's spinal alignment improves over the course of treatment, as assessed by repeat clinical imaging and updated Cobb angle measurements, the treating clinician may review updated diagnostic imaging and reconfigure the stimulation map of the stimulation garment 203 to reflect the evolving curvature profile, adjusting which electrodes are activated and the stimulation modality assigned to each, with the electrode activation pattern becoming progressively more symmetric as correction is achieved and the number of active electrode pairs on the convex side decreasing as the muscular imbalance is reduced. In some embodiments, the stimulation garment 203 may be configured for early-intervention use in patients presenting with mild curvatures below the conventional threshold for rigid bracing (for example, curvatures in the range of approximately 10-20 degrees Cobb angle), with the treating clinician prescribing a stimulation plan based on the diagnostic image 201 with the objective of intervening before the curvature progresses to a severity at which bone remodeling may result in a permanent structural deformity.

### Example Stimulation Garment—Compression Shirt with Embedded Electrodes

FIG. 3 illustrates an example stimulation garment 203 embodied as a compression shirt with embedded electrodes in accordance with one or more aspects of the present disclosure. As shown in FIG. 3, the stimulation garment 203 may comprise a compression shirt 215 configured to conform closely to the patient's torso, providing consistent electrode-to-skin contact across the thoracolumbar region without requiring a separate compressive wrap or external fastening mechanism. In some embodiments, the compression shirt 215 may be fabricated from a breathable, moisture-wicking, and stretchable material (e.g., a compression-grade synthetic stretch fabric) configured to maintain close apposition to the patient's skin throughout a range of body positions and daily activities. As further shown in FIG. 3, the stimulation garment 203 may include a plurality of first electrodes 217 and a plurality of second electrodes 219 embedded within the fabric of the compression shirt 215. In some embodiments, each of the first electrodes 217 and the second electrodes 219 may be selectively configured to operate as either a TENS electrode or an EMS electrode, such that the assignment of stimulation modality to each electrode is not fixed by the physical construction of the electrode but is instead determined by the treating clinician based on the patient's diagnosed orthopedic condition and the corresponding stimulation plan. In some embodiments, the first electrodes 217 and the second electrodes 219 may be textile electrodes woven or knitted into the fabric substrate of the compression shirt 215, or may be conductive elements affixed to the inner surface of the shirt 215 in predetermined positions corresponding to the target musculature. In some embodiments, electrodes positioned in proximity to the concave aspect of the patient's spinal curvature may be assigned to deliver transcutaneous electrical nerve stimulation configured to promote relaxation of the characteristically tight paraspinal musculature, while electrodes positioned in proximity to the convex aspect may be assigned to deliver electrical muscle stimulation configured to promote contraction and strengthening of the comparatively lax paraspinal musculature, in accordance with the stimulation plan described herein. In some embodiments, the assignment of the first electrodes 217 and the second electrodes 219 as TENS electrodes or EMS electrodes within the compression shirt 215 may be configurable by the treating clinician via the circuit device 115 or a companion application, enabling the clinician to modify which embedded electrodes deliver TENS stimulation and which deliver EMS stimulation based on the patient's diagnostic imaging and clinical assessment, without requiring physical repositioning of the electrodes within the garment.

In some embodiments, the contact sensors 221 may comprise resistive contact sensors, each including a pair of conductive traces disposed on the inner surface of the compression shirt 215 and configured such that when the patient's skin bridges the gap between the conductive traces, a measurable change in resistance is detected by the circuit device 115, indicating skin contact. In other embodiments, the contact sensors 221 may comprise capacitive proximity sensors configured to detect the presence of the patient's skin based on a change in capacitance at the sensor element caused by the dielectric properties of human tissue in proximity to the sensor, without requiring direct galvanic contact with the skin. In further embodiments, the contact sensors 221 may comprise impedance-based sensors configured to apply a low-level alternating current signal across a pair of electrodes and to measure the resulting bioimpedance, wherein a bioimpedance measurement within a predetermined range characteristic of human skin and subcutaneous tissue indicates that the stimulation garment 203 is in contact with the patient's body. In some embodiments, the contact sensors 221 may comprise thermistors or other temperature-sensing elements configured to detect the presence of body heat at the inner surface of the compression shirt 215, wherein a temperature reading within a range characteristic of human skin temperature indicates that the stimulation garment 203 is being worn. In some embodiments, the contact sensors 221 may comprise optical reflectance sensors, each including a light emitter and a photodetector disposed on the inner surface of the compression shirt 215 and configured to detect the reflectance characteristics of human skin, which differ from those of ambient air or fabric surfaces, thereby providing an indication of skin contact. In some embodiments, the contact sensors 221 may comprise galvanic skin response (GSR) sensors configured to measure the electrical conductance of the patient's skin, which varies with perspiration and sympathetic nervous system activity, wherein a conductance measurement above a baseline threshold indicates that the stimulation garment 203 is in contact with the patient's skin. In some embodiments, the stimulation garment 203 may incorporate two or more different types of contact sensors 221 to provide redundant and complementary skin-contact detection, reducing the likelihood of false-positive or false-negative contact determinations.

As further shown in FIG. 3, the stimulation garment 203 may include a plurality of contact sensors 221 distributed across the inner surface of the compression shirt 215 and configured to detect whether the stimulation garment 203 or compression shirt 215 is in contact with the patient's skin. In some embodiments, each contact sensor 221 may be configured to generate a signal indicative of skin contact or absence thereof, and the circuit device 115 may be configured to receive the signals from the plurality of contact sensors 221 and to determine, based on the received signals, whether the stimulation garment 203 is being worn by the patient at a given time. In some embodiments, the circuit device 115 may transmit the contact sensor data to a treating physician or clinician via the communication network 630 (FIG. 5), for example through the companion application or clinical monitoring platform, enabling remote monitoring of whether the stimulation garment 203 is being worn and whether adequate skin contact is maintained during prescribed treatment periods. In some embodiments, the circuit device 115 may use the aggregate contact sensor data to distinguish between the stimulation garment 203 being worn on the patient's body, resting on a surface, or being worn over an intervening layer of clothing that may impede electrode-to-skin conductivity, thereby supporting accurate compliance monitoring and ensuring that electrical stimulation is delivered only when adequate skin contact is confirmed.

### Example Process—Determining and Delivering an Optimal Electrical Stimulation Treatment Plan for Orthopedic Correction

FIG. 4 is a flowchart illustrating an example process 300 for determining and delivering an optimal treatment plan for a patient with an orthopedic condition, such as scoliosis, according to some embodiments. As described below, a particular implementation can omit some or all illustrated features/steps, may be implemented in some embodiments in a different order, and may not require some illustrated features to implement all embodiments. In some examples, an apparatus (e.g., processor 612 with memory 614) in connection with FIG. 5 can be used to perform example process 300. However, it should be appreciated that any suitable apparatus or means for carrying out the operations or features described below may perform process 300.

At step 312, process 300 can receive X-ray and clinical data from a patient in need of potential orthopedic correction. For example, process 300 may receive one or more X-ray images acquired by an X-ray machine 602, along with associated clinical information such as the patient's medical history, prior imaging results, age, body measurements, and any previously attempted treatment modalities. In some embodiments, this data may be retrieved from a clinical database 606 that stores patient records, images, and medical history, or may be received directly from the X-ray machine 602 via communication network 630 as described herein.

At step 314, process 300 can receive a diagnosis of an orthopedic condition from a provider. In some embodiments, the diagnosis may be provided by a licensed medical provider, such as an orthopedic surgeon, physiatrist, or physical therapist, and may include identification of the type, severity, and location of the orthopedic condition. For example, the diagnosis may identify an orthopedic condition such as scoliosis, including the degree of spinal curvature as measured by the Cobb angle, and may identify whether the curvature is an S-shaped or C-shaped curve.

At step 316, process 300 can determine an optimal electrode alignment for muscle stimulation to treat the diagnosed orthopedic condition. In some embodiments, this determination may be made by analyzing the received X-ray and clinical data to identify the trajectory and severity of the spinal curve, and identifying which muscle groups for example, the paraspinal musculature on the convex side of the curve may be targeted for stimulation. In some embodiments, the optimal electrode alignment may be determined at least in part by computing device 610 based on one or more machine learning models trained on clinical imaging data.

At step 318, process 300 can perform electrode placement calibration to verify electrode contact and alignment with the target musculature. In some embodiments, the calibration process may include applying a low-level electrical signal through the electrode array and measuring the resulting resistance or impedance at each electrode to determine which electrodes are in adequate contact with the patient's skin and underlying musculature, and which electrodes may be positioned over bony structures such as the spinal column. In some embodiments, the calibration may also include a physical movement routine performed by the patient for example, extending or flexing the torso while process 300 monitors electrode response to confirm correct positioning prior to initiating a treatment session.

At step 320, process 300 determines an optimal Transcutaneous Electrical Nerve Stimulation (TENS) and/or Electrical Muscle Stimulation (EMS) settings, including at least one of the following: frequency, pulse width, and intensity, to treat the diagnosed orthopedic condition. In some embodiments, the TENS settings and EMS settings are determined independently of one another, as each modality serves a distinct therapeutic purpose. In some examples, the TENS component is aimed to alleviate pain, while the EMS component is aimed to strengthen the paraspinal muscles. In some embodiments, example TENS settings may include a frequency in the range of approximately 50 Hz to 120 Hz and a pulse width in the range of approximately 50 microseconds to 100 microseconds, while example EMS settings may include a frequency in the range of approximately 30 Hz to 80 Hz and a pulse width in the range of approximately 200 microseconds to 500 microseconds. In some embodiments, the optimal settings may be determined by computing device 610 and transmitted to the stimulation array article 608 via communication network 630. In some embodiments, the TENS and EMS parameters may be adjusted through a graphical user interface (GUI) that allows a clinician or patient to input desired frequency and pulse width values, which are then sent to the device 100.

At step 322, the patient receives the prescribed treatment of muscle stimulation. In some embodiments, the patient wears the stimulation array article 608 (which also corresponds to stimulation garment 203), which delivers TENS and/or EMS therapy to the targeted muscle groups in accordance with the treatment parameters determined at step 320.

At step 324, process 300 can optionally monitor patient compliance and treatment data such as hours of use and stimulation delivery logs via a companion application or clinical monitoring platform. In some embodiments, the companion application may be installed on a mobile device such as a smartphone or tablet, and may communicate with the stimulation array article 608 via a short-range wireless connection such as Bluetooth Low Energy (BLE) to log treatment session data in real time. In some embodiments, the compliance data may be transmitted to a clinical database 606 or directly to a medical provider, enabling remote oversight of the patient's adherence to the prescribed treatment protocol. In some embodiments, process 300 may generate alerts to the patient, a caregiver, or a clinician if the patient's usage falls below a prescribed minimum threshold.

At step 326, process 300 includes a step of repeat clinical imaging and assessment of results. In some embodiments, repeat clinical imaging may include one or more follow-up X-rays or other imaging modalities to evaluate the patient's spinal alignment relative to prior imaging, assess any change in the Cobb angle, and determine the patient's response to the prescribed treatment. In some embodiments, the repeat imaging data may be received by computing device 610 from X-ray machine 602 or clinical database 606 and compared against the baseline imaging data received at step 312 to quantify treatment progress.

In some embodiments, based on the assessment of the repeat clinical imaging, the method 300 proceeds along one of three branches: Branch 326A, Branch 326B, or Branch 326C.

Under Branch 326A, if the patient appears to be in normal alignment and no further treatment is necessary, process 300 proceeds to discontinue treatment and terminates. In some embodiments, a determination of normal alignment may be made by a medical provider based on a review of the repeat clinical imaging, optionally assisted by the computing device 610. In some embodiments, the patient may be discharged from active treatment and placed on a monitoring protocol to confirm maintained alignment over time.

Under Branch 326B, if modification of the treatment plan is needed, process 300 can adjust the pattern and/or strength of electrode stimulation. In some embodiments, modifying the treatment plan may include adjusting which electrodes are active, changing the TENS and/or EMS frequency, pulse width, or intensity, or repositioning the electrode array to better target the relevant musculature based on the updated imaging data. Following modification of the treatment plan, process 300 loops back to step 326 to perform repeat clinical imaging and assess results, thereby establishing an iterative feedback loop between clinical imaging and treatment adjustment until the patient achieves satisfactory alignment or a determination is made to discontinue treatment.

Under Branch 326C, if no modification is needed and the subject remains under the current treatment protocol, process 300 loops back to step 322 to continue the patient's prescribed treatment, with ongoing compliance monitoring at step 324 and periodic repeat clinical imaging at step 326 to assess continued treatment progress.

At step 328, if the repeat clinical imaging and assessment of results indicate that the treatment plan should be modified, process 300 can adjust the treatment plan accordingly, including modifying the TENS and/or EMS parameters, electrode alignment, or stimulation pattern, before returning to the repeat clinical imaging step to evaluate the effect of the modified treatment plan.

### Example Hardware Integration of Model

FIG. 5 shows a block diagram illustrating a system 600 for determining an optimal treatment plan for a patient with an orthopedic condition, such as scoliosis, and for delivering and adjusting a prescribed course of electrical muscle stimulation and/or transcutaneous electrical nerve stimulation, according to some embodiments.

As shown in FIG. 5, computing device 610 can receive clinical imaging data (e.g., X-ray images) and other patient data from an X-ray machine 602 and/or from a clinical database 606, which stores patient records, images, and medical history. Computing device 610 can additionally receive a diagnosis of an orthopedic condition, such as scoliosis, from a medical provider. Computing device 610 can process the received imaging data and clinical information to determine an optimal electrode alignment for muscle stimulation based on the diagnosed orthopedic condition, and to determine optimal electrical stimulation settings for treating the patient. Alternatively, computing device 610 may provide a user interface displaying a mock spine (or an image of the patient's spine) and: guide a provider to input desired locations for stimulation as part of a stimulation plan; display a proposed stimulation location plan allowing for provider adjustment and supplementation; or accept user input as text or voice and interpret (e.g., via an LLM or vision-language model) a suggested stimulation plan to be displayed. Computing device 610 can additionally communicate treatment parameters to memory as part of a stimulation plan and/or to a stimulation array article 608, which is configured to deliver electrical muscle stimulation (EMS) and/or transcutaneous electrical nerve stimulation (TENS) to targeted muscle groups of the patient. In some embodiments, the stimulation array article 608 may comprise a wearable article incorporating an array of electrodes such as the silver fabric electrode pads described with respect to the device 100 of FIGS. 1A-1C that can be selectively activated to deliver stimulation to the paraspinal or other relevant musculature in accordance with the treatment plan determined by process 300 (FIG. 4). In some embodiments, the stimulation array article 608 may correspond to the stimulation garment 203 described with respect to FIG. 2, and may embody the device 100 described with respect to FIGS. 1A-1C, including the electrode pair positions, compressive wrap portion 107, lead wires 109, and circuit device 115 described herein. In some embodiments, the stimulation array article 608 corresponds to the stimulation garment 203.

In some embodiments, the electrode array comprises four channels of electrically conductive electrodes for example, silver/silver chloride fabric electrode patches distributed bilaterally along the paraspinal musculature, with two channels positioned on each side of the spinal column. In other embodiments, the electrode array may comprise more or fewer channels (e.g., two, six, eight, or more) depending on the patient's anatomy and the extent of spinal curvature to be addressed.

In some examples, computing device 610 can include processor 612. In some embodiments, the processor 612 may be any suitable hardware processor or combination of processors, such as a central processing unit (CPU), a graphics processing unit (GPU), a neural processing unit (NPU), a tensor processing unit (TPU), an application-specific integrated circuit (ASIC), a field-programmable gate array (FPGA), a digital signal processor (DSP), a microcontroller (MCU), a system-on-chip (SoC), a neuromorphic processor, or any other suitable processing unit or combination thereof. For example, the ASIC may be purpose-built to implement a specific machine learning model or inference pipeline. In embodiments involving edge deployment, low-power inference, or miniaturized form factors (e.g., wearable devices, remote or field-installed devices, or embedded edge or IoT processors), the processor 612 may be a resource-constrained microcontroller or a dedicated low-power AI accelerator optimized for on-device inference with minimal energy consumption. In embodiments involving high-throughput analyses, large-scale data processing, or large-scale parallel inference, the processor 612 may comprise one or more GPUs, TPUs, or distributed processing clusters. The processor 612 may reflect general-purpose computational resources, or may be dedicated processing resources optimized for a specific function contemplated herein. The processor 612 may be located within a local device, may be associated with a system hosting a patient medical record application, may be associated with a system providing information to clinicians, may be part of a cloud-based resource, or otherwise, depending on the particular embodiment.

In further examples, computing device 610 can further include a memory 614. The memory 614 can include any suitable storage device or devices that can be used to store suitable data (e.g., software, model weights, firmware, user settings, sensor data, and any other data or information used in performing the processes contemplated herein). The memory 614 can include a non-transitory computer-readable medium comprising any suitable volatile memory, non-volatile memory, storage, or any suitable combination thereof. For example, memory 614 can include random access memory (RAM), read-only memory (ROM), electronically-erasable programmable read-only memory (EEPROM), one or more flash drives, one or more hard disks, one or more solid-state drives (SSDs), one or more optical drives, embedded flash memory, or on-chip memory integrated with the processor. In resource-constrained embodiments, memory 614 may be limited in capacity and optimized for low-power retention of model parameters and inference outputs.

In further examples, computing device 610 can further include communications system 618. The communications system 618 can include a variety of suitable hardware, firmware, and/or software for communicating information over one or more communication networks (in parallel, in series, and/or mixtures thereof, and whether similar or heterogeneous), which may include local, wide-area, private, public, or hybrid networks, and which may involve communication among a plurality of heterogeneous devices, nodes, servers, and cloud resources communicating on similar or different protocols and timing. For example, communications system 618 can include one or more transceivers, one or more communication chips and/or chip sets, antennas, modems, and associated signal processing components. In a more particular example, communications system 618 can include hardware, firmware, and/or software that can be used to establish a Wi-Fi connection (including IEEE 802.11 ax/Wi-Fi 6 or Wi-Fi 6E), a Bluetooth or Bluetooth Low Energy (BLE) connection, a cellular connection (e.g., a 4G LTE, 5G NR, or future-generation network complying with any suitable standard, such as CDMA, GSM, LTE Advanced, NR, or mmWave 5G), a satellite connection (including low-earth orbit (LEO) satellite networks), a Zigbee, Z-Wave, Thread, or other low-power mesh network connection, a near-field communication (NFC) connection, a LoRa or LoRaWAN connection for long-range low-power applications, a wired Ethernet connection, or any combination thereof. In some embodiments, the computing device 610 may participate in a multi-tier architecture in which data is communicated among on-device edge processing, local fog or gateway nodes, and remote cloud servers or data centers, with different processing tasks allocated across tiers based on latency, bandwidth, privacy, and computational resource constraints.

In further examples, computing device 610 can receive or transmit information (e.g., from or to an X-ray machine 602, a facility, clinic, or hospital 604, a clinical database 606 storing patient records, images, and medical history, a stimulation array article 608, and/or any other suitable system or device) over a communication network 630. In some examples, communication network 630 can be any suitable communication network or combination of communication networks. For example, communication network 630 can include a Wi-Fi network, a peer-to-peer network (e.g., a Bluetooth or BLE network), a cellular network (e.g., a 4G LTE, 5G NR, or future-generation network), a wired network, or similar. In some embodiments, communication network 630 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, or similar.

In some examples, computing device 610 can further transmit an output connection 622 to an X-ray machine 602, a facility, clinic, or hospital 604, a clinical database 606, or a stimulation array article 608. The output connection 622 may be part of or rely upon communication network 630, but alternatively may be a separate connection, such as a private connection to a healthcare organization's electronic medical record system, or may include other connections such as an email server. In some embodiments, the output connection 622 can transmit a treatment plan, adjusted stimulation settings, a compliance report, or an alert to a medical provider, patient, or third party based on the patient's clinical imaging results and response to treatment.

In further examples, computing device 610 can further include a display 616 and/or one or more inputs 620. In some embodiments, the display 616 may include any suitable combination of input and output components for interaction between the system and a user, operator, clinician, or other human. For example, the display 616 may include any suitable display devices, such as a native dashboard, display screen, touchscreen, kiosk, liquid crystal display (LCD), infotainment screen, organic light-emitting diode (OLED) display, an e-ink display, an augmented reality (AR) or mixed reality (MR) overlay, or a heads-up display (HUD), to present and/or obtain information, alerts, visualizations, or diagnostic outputs to or from a user at various points in performance of the methods described herein. In some embodiments, the display 616 may additionally or alternatively include audio output components (e.g., speakers or haptic actuators) and audio or gesture-based input components (e.g., microphones, voice recognition interfaces, or motion sensors) to support hands-free or accessibility-oriented interaction. In further embodiments, the input(s) 620 can include any suitable input devices and/or sensors that can be used to receive user input, such as a keyboard, a mouse, a touchscreen, a microphone, or similar.

In embodiments directed to wearable devices or other miniaturized form factors, the user interface may be minimal or absent entirely, with outputs communicated instead to a companion mobile application, a clinical monitoring platform, or a remote dashboard accessible via a network connection. In such embodiments, a separate computing device (e.g., a smartphone, tablet, or clinical workstation) may serve as the primary user-facing interface for the system, enabling a clinician or patient to review treatment progress, compliance data, adjusted stimulation parameters, and imaging results without direct interaction with the computing device 610 itself.

### Further Embodiments, Implementations, and Applications

The foregoing descriptions of example methods, hardware configurations, and validation studies including characterization of the TENS and EMS output voltage and current characteristics of the circuit device 115, verification of electrode-to-skin contact across representative body morphologies, verification of stimulation delivery to intended anatomical targets, printed circuit board design and simulation of the electronic subsystem, and integration of the electronic subsystem with the wearable orthotic form factor serve both as written description of their particular subject matter and contemplated embodiments as well as more general enablement of underlying operational principles, functionality, techniques, improvements, and other features. Although the validation studies were performed using certain implementations and configurations, it should be understood that the results of these studies are indicative of the advantages of all contemplated embodiments; the particular examples used for purposes of the validation studies are not intended as limiting. The experiments described below serve as illustrative examples of the improvements achieved by embodiments of the present disclosure. Following the validation studies, the present disclosure will describe several additional embodiments and implementations in which some or all of such features may be leveraged to achieve new, improved, tangible, and useful results in particular fields of application.

In accordance with the foregoing, the inventors conducted validation testing of the device 100 described herein, including characterization of the TENS and EMS output voltage and current characteristics of the circuit device 115, verification of electrode-to-skin contact across representative body morphologies, verification of stimulation delivery to intended anatomical targets, printed circuit board (PCB) design and simulation of the electronic subsystem, and integration of the electronic subsystem with the wearable orthotic form factor.

In a representative proof-of-principle contact verification study, a wearable wrap prototype comprising eight silver/silver chloride fabric electrode contact points was applied to subjects representing a range of body morphologies, including pediatric and adult subjects and subjects with varying degrees of trunk musculature and body composition including muscular, uneven, round, flat, firm, and forward-curved back morphologies, as well as a 9.5-year-old male subject and a 21-year-old female subject. To verify electrode-to-skin contact, an adhesive tape transfer method was employed: adhesive tape was applied to each electrode surface, the electrode array was pressed against the subject's skin, and the tape was peeled back to confirm that conductive material had transferred, thereby confirming contact at each electrode location. All eight electrode contacts (8 out of 8) were confirmed using this method across all tested subjects, demonstrating the feasibility of the silver/silver chloride fabric electrode approach for achieving reliable and verifiable electrode-to-skin contact across a representative range of patient body types and morphologies.

To characterize the output voltage and current delivered by the circuit device 115, the TENS and EMS stimulation channels were each tested across a range of simulated skin resistances representative of the impedance variation expected across patient populations. Table 2 summarizes the average voltage and current measured at the TENS output across three simulated skin resistance values (500 Ω, 2 kΩ, and 10 kΩ), demonstrating consistent voltage output across the tested impedance range.

TABLE 2
Table recording average voltage and current
for TENS at stimulated skin resistances.

| Resistances | | Average | Average |
|---|---|---|---|
| (Ω) | Voltage (V) | Voltage (V) | Current (mA) |
| 500 | 44.7, 44.5, 44.4, 44.4, | 44.4 | 88.8 |
| | 44.2, 44.2, 44.5, 44.5, | | |
| | 44.4, 44.4 | | |
| 2K | 45.9, 45.6, 45.7, 45.6, | 46.1 | 23.1 |
| | 45.6, | | |
| | 46.5, 46.5, 46.4, 46.4, | | |
| | 46.4 | | |
| 10K | 44.8, 45.0, 44.9, 44.9, | 44.9 | 4.49 |
| | 45.0, 44.9, 45.0, 44.9, | | |
| | 44.9, 45.0 | | |

Table 3 summarizes the corresponding measurements for the EMS output across the same simulated skin resistance values. As with the TENS channel, the EMS output exhibited stable average voltage across the tested impedance range, confirming consistent stimulation delivery under varying load conditions representative of electrode-to-skin interface variability encountered in clinical use.

TABLE 3
Table recording average voltage and current
for EMS at simulated skin resistances.

| Resistances | | Average | Average |
|---|---|---|---|
| (Ω) | Voltage (V) | Voltage (V) | Current (mA) |
| 500 | 40.2, 40.8, 40.4, | 40.2 | 80.4 |
| | 40.0, 39.8, 40.0 | | |
| 2K | 38.1, 38.6, 38.6, | 38.7 | 19.4 |
| | 38.8, 38.9, 39.2 | | |
| 10K | 38.7, 39.3, 38.4, | 39.2 | 3.92 |
| | 40.0, 39.9, 38.8 | | |

To demonstrate the generalizability of the content of the present disclosure to a variety of practical applications, the present disclosure will now describe several additional embodiments and implementations in which some or all of the foregoing features may be leveraged to achieve new, improved, tangible, and useful results in particular fields of application.

In one such implementation, the present disclosure contemplates a clinic-configured therapeutic garment embodying the wearable orthopedic muscle stimulation device 100 described with respect to FIGS. 1A-1C. During an initial clinical visit, a physical therapist or orthotist may fit the wearable wrap to the patient, position the electrode array at anatomically determined stimulation targets corresponding to the patient's spinal curvature profile, and configure stimulation parameters via the device interface. Electrode contact quality may be verified prior to the first session. The patient may be trained in donning, doffing, and operating the device independently for home use. In some embodiments, the commercial product may include a soft, adjustable wearable wrap with integrated electrodes, a compact electronics module, a rechargeable battery pack, and a companion mobile application. The application may provide a practitioner-facing dashboard for prescribing stimulation schedules, reviewing session logs, adjusting parameters remotely, and prompting periodic in-clinic recalibration.

In some embodiments, a clinician such as a physical therapist applies one or more adhesive electrode elements (e.g., pre-gelled adhesive electrode stickers similar to those used in electrocardiography) directly to the patient's skin at anatomically determined locations during an initial clinical visit. The wearable garment 100 is then positioned over the adhesive electrode elements such that the conductive electrode elements within the garment make electrical contact with the adhesive stickers upon donning. This hybrid approach ensures that electrode-to-skin contact is maintained at clinician-determined positions across successive at-home treatment sessions, without requiring the patient to independently locate and re-apply electrodes to precise anatomical positions. In some embodiments, the adhesive electrode elements are replaceable and disposable, and the patient is provided with replacement elements to be positioned at the same clinician-marked locations for subsequent treatment sessions.

In another implementation, the present disclosure provides for a wearable therapeutic compression shirt designed to extend electrode coverage across the full thoracolumbar region, enabling stimulation targets higher on the spine than a lumbar band or wrap can reach. An electrode array comprising a plurality of individually addressable elements may be integrated into the inner surface of the shirt garment, with lead wires routed to a compact controller module. The controller may selectively activate subsets of electrodes for TENS, EMS, or combined stimulation based on a stimulation map corresponding to the patient's curvature pattern. The garment may be constructed from breathable, moisture-wicking materials compatible with extended wear and adjustable to accommodate a range of thoracolumbar body dimensions. The electronics module may be detachable for laundering and charging. This implementation may be particularly suited for patients with double-curve (S-shaped) scoliosis requiring simultaneous stimulation at multiple spinal levels.

In a further implementation, the system may incorporate an electrode-to-skin impedance sensing subsystem that actively maps which electrodes in the array are in adequate contact with the patient's skin, and derives from this mapping an approximation of the trajectory of the patient's spine and the location of anatomical landmarks such as the spinous processes and medial furrow. The impedance sensing circuit may apply a low-level test signal to each electrode in sequence and measure the resulting impedance, flagging electrodes that exceed a contact-quality threshold. The resulting contact map may be processed by the controller to identify the path of the spine and to adjust the stimulation plan accordingly, activating only those electrodes that are in adequate contact and within appropriate anatomical proximity to the target muscles. A visual representation of the contact map may be displayed to the clinician or patient, enabling guided repositioning of the garment. This implementation may accommodate patient-to-patient variability in body composition, including subcutaneous fat, perspiration, and skin texture.

In yet another implementation, the system may provide a patient-facing guided calibration workflow within a companion mobile application to enable proper device alignment and electrode positioning without direct clinical supervision. Upon donning the device, the patient may be prompted to perform a series of brief guided movements. Such movements may include, for example, standing straight, bending forward, and returning to neutral posture. During these movements, the device's sensors and electrode contact data are used to verify proper garment positioning and to re-zero the alignment reference. The application may provide real-time feedback through on-screen indicators or haptic cues indicating whether the garment is correctly positioned or requires adjustment. Calibration may be triggered automatically at each session start or on a scheduled periodic basis as the patient's body morphology evolves over the treatment course.

In another implementation, the system may be implemented as a connected digital health platform in which the wearable device 100 communicates session data to a cloud-based data management service. Such session data may include time worn, stimulation modalities delivered, electrode contact status, battery level, and stimulation intensity settings. A clinician-facing web dashboard may present this data in aggregated and per-session views, enabling monitoring of patient compliance and review of trends in session quality. The platform may support remote adjustment of stimulation parameters transmitted to the device 100 via a companion application. Integration with a patient's electronic medical record system may enable automatic documentation of session data and support compliance records relevant to reimbursement.

In a further implementation, the system may be deployed as an early-intervention therapeutic device for patients presenting with mild AIS at curvature levels below the conventional threshold for rigid brace prescription. The primary therapeutic goals in this context may be to inhibit further curve progression through targeted paraspinal muscle activation and to address neuromusculoskeletal contributors to curvature development, without the compliance challenges associated with rigid bracing. The device 100 may be prescribed as part of a conservative care regimen including physical therapy and monitored activity. The system may also function as an adjuvant pain management and compliance-enhancement tool for patients concurrently transitioning to or wearing a rigid brace, potentially delaying or reducing the need for full-time rigid bracing.

In another implementation, the embodiments described with respect to FIGS. 1A-1C may be integrated with non-radiological spinal curvature monitoring modalities to supplement or reduce reliance on periodic radiographic imaging, thereby limiting cumulative radiation exposure particularly for the pediatric patient population. In some embodiments, treatment progress is monitored using non-radiologic assessment methods, alone or in combination with periodic radiographic imaging, to reduce cumulative radiation exposure a consideration of particular clinical significance in the pediatric and adolescent patient populations most commonly affected by AIS. Non-radiologic monitoring modalities may include, without limitation: (i) three-dimensional motion capture systems configured to track dynamic spinal movement and posture during standardized activities; (ii) surface topography scanning to assess the external contour of the patient's trunk as a surrogate for underlying spinal curvature; (iii) clinical physical examination including the Adams forward-bend test, which assesses rib cage rotation as a clinical indicator of rotational spinal curvature; (iv) longitudinal postural data collected by one or more IMUs integrated into the wearable device, used as a continuous non-radiologic proxy measure for changes in spinal alignment over the course of treatment; and (v) emerging low-dose radiographic technologies, which may substantially reduce per-examination radiation dose relative to conventional spinal X-ray. In some embodiments, the companion application aggregates IMU-derived postural data over successive treatment sessions and presents longitudinal postural trend data to the clinician through the clinical platform interface, enabling non-radiologic tracking of treatment response between scheduled clinical imaging appointments. In some embodiments, the garment's electrode array and sensor suite may be used in conjunction with external motion-capture systems or surface-topography scanning tools to derive a dynamic profile of spinal curvature based on observable back-surface movement and posture. Stimulation may be triggered and observed during monitoring sessions to correlate therapeutic response with positional data, providing a richer characterization of therapeutic effect than static imaging alone.

In yet another implementation, the electronic stimulation subsystem of the device 100 described with respect to FIGS. 1A-1C comprising the stimulation circuit, electrode array interface, controller module, and firmware may be offered as a modular component for integration into the product lines of established spinal care orthotic manufacturers. An orthotic manufacturer incorporating the stimulation technology described herein may integrate the stimulation electronics into existing brace form factors, such as rigid or semi-rigid orthoses, as an optional or standard therapeutic enhancement feature. In this implementation, the modular subsystem may be designed for compatibility with standard electrode connector interfaces and may include a configuration application programming interface accessible to the manufacturer's companion software. This implementation may be particularly attractive to manufacturers that maintain existing relationships with orthopedic surgeons, physiatrists, and physical therapists who prescribe spinal orthoses, as the stimulation module may be offered as a value-added option within the manufacturer's existing distribution and clinical support channels, combining the manufacturer's expertise in brace fabrication and fitting with the active neuromuscular stimulation capabilities of the device 100.

In a further implementation, the system described with respect to FIGS. 1A-1C may be commercially positioned for adoption within the neuromuscular rehabilitation market, addressing patients who develop scoliosis or spinal asymmetry secondary to a neurological event such as stroke, traumatic spinal cord injury, or a degenerative neuromuscular condition. In this implementation, the imaging-driven electrode activation methodology and dual-modality TENS and EMS delivery of the device 100 may be applied to patients whose spinal curvature arises from unilateral or asymmetric loss of neuromuscular control, rather than from idiopathic developmental causes. The system may be distinguished from existing neuromuscular rehabilitation platforms in that the electrode activation pattern is derived from spinal curvature imaging data and is specifically configured to address the concave-convex muscular imbalance characteristic of scoliotic deformity, rather than providing generalized whole-body or compartmentalized muscle stimulation. In some embodiments, the device 100 may be prescribed as a component of a broader neurorehabilitation program, with the scoliosis-specific stimulation protocol operating in coordination with other rehabilitative interventions prescribed by the patient's care team. This implementation may expand the addressable patient population for the technology described herein beyond adolescent idiopathic scoliosis to include adult and geriatric patients presenting with acquired spinal deformity secondary to neurological injury or age-related musculoskeletal degeneration.

In yet another implementation, the system described with respect to FIGS. 1A-1C may be commercially deployed for use in supervised clinical treatment sessions administered by a physical therapist or other licensed practitioner, in addition to or as an alternative to at-home wearable use. In this implementation, the treating clinician may apply the electrode array of the device 100 to the patient at each clinical visit, configure the stimulation parameters based on the most recent clinical imaging, and supervise the treatment session in real time. The clinician-configurable stimulation mapping and imaging-derived electrode activation protocol of the device 100 may be particularly suited to this supervised deployment model, as the clinician may adjust the stimulation plan at each visit based on observed clinical response and updated imaging without requiring the patient to manage device configuration independently. In some embodiments, the supervised clinical deployment may be used during an initial treatment phase to establish baseline stimulation parameters and verify therapeutic response before transitioning the patient to an at-home wearable regimen, or may be used on a periodic basis to deliver higher-intensity supervised sessions that complement lower-intensity daily at-home stimulation. This implementation may support reimbursement under existing procedural billing codes for neuromuscular reeducation or therapeutic electrical stimulation, facilitating clinical adoption.

In another implementation, the system described with respect to FIGS. 1A-1C may be commercially deployed in conjunction with prescribed physical therapy exercise regimens, such as Schroth method exercises or core stabilization routines, to augment the therapeutic effect of active movement with concurrent targeted paraspinal muscle stimulation. In this implementation, the patient may wear the device 100 during supervised or at-home exercise sessions, with the device delivering scoliosis-specific EMS to the convex-side paraspinal musculature and TENS to the concave-side musculature while the patient performs prescribed corrective exercises. The system may be distinguished from general-purpose exercise-enhancement stimulation products in that the electrode activation pattern and stimulation modality assignment are derived from the patient's spinal curvature imaging data and are specifically configured to address the muscular imbalances associated with the diagnosed scoliotic deformity, rather than providing uniform or compartmentalized stimulation for general fitness purposes. In some embodiments, the companion application may include an exercise-session mode that coordinates stimulation delivery with prescribed exercise timing, and may log exercise-session compliance data alongside stimulation-session data for review by the treating clinician.

In a further implementation, the system described with respect to FIGS. 1A-1C may be configured to accommodate supplemental therapeutic modalities in addition to TENS and EMS stimulation. For example, in some embodiments, the wearable garment may include provisions for integrating additional therapeutic modality elements, such as near-infrared or far-infrared emitters, positioned in proximity to the target paraspinal musculature. Such supplemental modalities may be controlled independently of, or in coordination with, the TENS and EMS stimulation channels, and may be configured by the treating clinician to promote local tissue perfusion, reduce inflammation, or support muscular recovery in the stimulated regions. The circuit device 115 may provide additional output channels or accessory ports to accommodate such supplemental therapeutic elements without requiring modification of the core stimulation circuitry. This implementation may provide a multi-modal therapeutic platform within a single wearable form factor, with the scoliosis-specific TENS and EMS stimulation protocol remaining the primary therapeutic modality and supplemental modalities serving an adjunctive role as clinically indicated.

In yet another implementation, the system described with respect to FIGS. 1A-1C may be commercially deployed for geriatric patients presenting with spinal curvature or postural asymmetry secondary to age-related musculoskeletal degeneration, including degenerative disc disease, vertebral compression fractures, or progressive loss of paraspinal muscle tone. In this implementation, the imaging-driven stimulation protocol of the device 100 may be adapted to address the particular characteristics of degenerative spinal curvature, which may differ from adolescent idiopathic scoliosis in etiology, rate of progression, and therapeutic objectives. The primary therapeutic goals in the geriatric context may include maintenance of existing spinal alignment, strengthening of weakened paraspinal musculature to resist further postural deterioration, and management of chronic pain associated with the degenerative condition. In some embodiments, the stimulation parameters may be configured at lower intensities appropriate for the geriatric patient population, and the companion application may include caregiver-facing monitoring features enabling remote oversight of treatment compliance and device status. This implementation may expand the commercial addressable market for the technology described herein to include the substantial and growing population of older adults presenting with symptomatic spinal degeneration.

In another implementation, the system described with respect to FIGS. 1A-1C may be developed and commercialized as a prescription digital therapeutic (PDT): a device-based therapeutic intervention that may be subject to regulatory review and eligible for third-party reimbursement as a covered medical device benefit. In this embodiment, the system may be marketed to health insurers, employer health benefit programs, and integrated health systems as a clinically-validated, device-delivered rehabilitation intervention for scoliosis and related spinal deformities. A medical device company utilizing the systems and methods disclosed herein may obtain regulatory clearance or approval for a product incorporating the wearable stimulation platform of the device 100, clinical outcome tracking and data interpretation capabilities, provider oversight tools, and patient-reported outcome modules. The reimbursement pathway may leverage existing procedural billing codes for neuromuscular reeducation, therapeutic electrical stimulation, or orthotic device management, or may pursue a novel reimbursement classification specific to integrated stimulation-orthotic devices for spinal deformity correction.

In some embodiments, the wearable garment is configured as a wearable shirt or vest providing electrode coverage spanning from the upper thoracic spinal region (e.g., at or above the T1-T7 vertebral levels) through the lumbar region. Such a configuration may be advantageous in cases where the spinal curvature has a thoracolumbar or thoracic primary curve component, where a lumbar wrap alone would not provide electrode access to the relevant paraspinal musculature at upper thoracic levels. In some embodiments, the wearable shirt is configured to be worn beneath a conventional outer garment, promoting patient comfort and long-term compliance, particularly in pediatric and adolescent patient populations.

In some embodiments, a wearable orthopedic stimulation device may be used prophylactically in patients presenting with signs of early-stage or mild spinal curvature that does not yet meet the threshold for prescription of a conventional rigid orthopedic brace (e.g., Cobb angle below approximately 20-25 degrees). In such embodiments, a provider may not yet have diagnosed a musculoskeletal disease, but has some reason to believe the patient is susceptible to developing the disease. Accordingly, a stimulation plan may not be as aggressive in forcing major curvature changes: lower levels of stimulation may be used, fewer electrodes used, smaller electrodes used, and/or electrode placement design may be modified to account for lesser or no existing curvature. Thus, a device may be used to address neuromusculoskeletal imbalances associated with the early-stage curvature before the curvature becomes structural, with goals of preventing, slowing, halting, or reversing curvature progression. In some embodiments, the device is further used prophylactically in patients identified as being at elevated risk of developing scoliosis due to neurological conditions, prior musculoskeletal injury, or developmental factors.

In some embodiments, a wearable orthopedic stimulation device is used as an adjuvant therapy alongside or integrated into a conventional rigid orthopedic brace or orthotic device. Rigid orthopedic braces prescribed for scoliosis management (e.g., TLSO/Boston brace) are typically recommended to be worn for 16-23 hours per day; however, patient compliance is frequently poor, particularly among adolescent patients, due to discomfort, aesthetic concerns, and interference with daily activities. In such embodiments, the conventional brace may be worn for fewer hours per day, with a wearable orthopedic stimulation device configured to deliver TENS to provide continued adjustment during additional hours. This dual approach can thereby promoting improved patient compliance with the prescribed bracing regimen. In some embodiments, the wearable device is integrated within the rigid brace or configured as a thin/tight wrap or article that can be positioned beneath it, for wearing all day whether the conventional brace is on or not.

In some embodiments, an impedance measurement calibration process is used not only to verify electrode-to-skin contact (or simply sufficient conductive contact), but also to approximate the trajectory of the patient's spinal column or changes therein. Electrodes positioned directly over bony prominences (such as the vertebral spinous processes) may exhibit higher impedance compared to electrodes positioned over adjacent paraspinal musculature, due to differences in tissue conductivity. In a patient with spinal curvature, a pattern of high-impedance being detected by a contact array could be interpreted as corresponding to the vertebral spine or midline spinous process divot. Row/column information associated with such contacts can be used to localize and define a non-straight path across the electrode array, thereby providing an impedance-derived approximation of the spinal curvature trajectory. In some embodiments, this impedance-derived trajectory is used to assign electrode contacts to concave-side and convex-side zones for differential TENS and EMS delivery, supplementing or reducing reliance on clinical imaging data.

In some embodiments, the wearable orthopedic stimulation device further comprises one or more infrared therapy emitters configured to deliver infrared radiation to the patient's paraspinal musculature. Infrared therapy may be delivered in conjunction with or independently of the electrical stimulation modalities (TENS and/or EMS), and may provide additional therapeutic effects including pain relief, promotion of local circulation, and muscle relaxation. The infrared emitters may be integrated into the wearable garment substrate alongside the electrode array, and may be utilized on opposing sides of the spine from the electrodes providing stimulation.

In some embodiments, the plurality of electrodes are organized into a series of source-sink electrode pairs arranged along the trajectory of the target paraspinal musculature, forming a series of microcircuits along the length of the curvature. In some embodiments, a greater number of source-sink microcircuits are activated on the convex side of the spinal curvature (to promote
greater EMS-driven muscle contraction and strengthening) relative to the number of microcircuits activated on the concave side (where TENS-driven relaxation is the primary modality). In some embodiments, the balance between the number of active microcircuits on the convex versus concave sides is tracked over successive treatment sessions as a measure of corrective progress, and may be adjusted by the clinician through the clinical platform interface as the patient's curvature responds to treatment

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

What is claimed is:

1. A method for treating an orthopedic condition affecting a musculoskeletal system of a patient, the method comprising:
determining a stimulation plan for a wearable orthopedic stimulation device worn on the patient, the stimulation plan specifying activation of a plurality of electrodes of the wearable orthopedic stimulation device and assignment of at least one electrical stimulation modality to each activated electrode based on a location of the activated electrode relative to target musculature of the patient associated with the orthopedic condition; and
delivering, via the wearable orthopedic stimulation device, electrical stimulation to the target musculature in accordance with the stimulation plan.

2. The method of claim 1, wherein determining the stimulation plan comprises:
receiving clinical imaging data of the patient; and
based at least in part on the clinical imaging data, determining which electrodes of the plurality of electrodes are to be activated.

3. The method of claim 2, wherein determining the stimulation plan further comprises assigning at least one of transcutaneous electrical nerve stimulation (TENS) and electrical muscle stimulation (EMS) to each activated electrode based on a location of the activated electrode relative to the target musculature.

4. The method of claim 3, wherein the orthopedic condition comprises scoliosis, and wherein determining the stimulation plan further comprises:
identifying, from the clinical imaging data, a concave aspect and a convex aspect of a spinal curvature of the patient;
assigning electrodes positioned in proximity to the convex aspect to deliver EMS configured to promote contraction and strengthening of paraspinal musculature located at the convex aspect of the spinal curvature; and
assigning electrodes positioned in proximity to the concave aspect to deliver TENS configured to promote relaxation of paraspinal musculature at the concave aspect.

5. The method of claim 3, wherein the clinical imaging data comprises at least one X-ray image from which one or more Cobb angle measurements characterizing a degree and location of lateral spinal curvature of the patient are determined, and wherein the stimulation plan is based at least in part on the one or more Cobb angle measurements.

6. The method of claim 1, wherein the wearable orthopedic stimulation device is prescribed for use in a patient presenting with early-stage neuromusculoskeletal markers of the orthopedic condition.

7. The method of claim 6, wherein the early-stage neuromusculoskeletal markers comprise a spinal curvature having a Cobb angle measurement of approximately 10 to 25 degrees.

8. The method of claim 1, further comprising performing a stimulation placement calibration prior to delivering the electrical stimulation, the stimulation placement calibration comprising:
applying a test signal through the plurality of electrodes;
measuring a resulting impedance at each electrode of the plurality of electrodes; and
determining, based on the measured impedance, which electrodes of the plurality of electrodes are in conductive contact with the patient at a position overlying the target musculature.

9. The method of claim 8, wherein the stimulation placement calibration further comprises deriving, from the measured impedance, an approximation of a spinal trajectory of the patient by identifying electrodes overlying bony structures based on impedance values exceeding a contact-quality threshold, and wherein the stimulation plan is further based on the derived spinal trajectory approximation.

10. The method of claim 1, further comprising monitoring patient compliance with a prescribed treatment protocol, wherein monitoring patient compliance comprises receiving, by a companion application from the wearable orthopedic stimulation device via a wireless communication link, session data comprising at least one of: time worn, stimulation modalities delivered, electrode contact status, and stimulation intensity settings, and transmitting the session data to a clinical database accessible by a medical provider.

11. The method of claim 10, wherein monitoring patient compliance further comprises detecting, via an inertial measurement unit integrated into a wearable garment of the wearable orthopedic stimulation device and communicatively coupled to a circuit device thereof, an orientation and movement of the wearable garment to determine whether the wearable garment is being worn by the patient during a given period.

12. The method of claim 1, further comprising:
performing repeat clinical imaging of the patient after a period of treatment;
assessing a change in the orthopedic condition based on the repeat clinical imaging; and
based on the assessment, selectively:
discontinuing treatment upon determination that the patient has achieved satisfactory musculoskeletal alignment;
modifying the stimulation plan based on the repeat clinical imaging and returning to delivering electrical stimulation in accordance with the modified stimulation plan; or
continuing treatment according to a prescribed protocol without modification.

13. The method of claim 1, wherein the wearable orthopedic stimulation device is used as an adjuvant therapy alongside a conventional rigid orthopedic brace worn by the patient, the electrical stimulation providing pain relief and neuromuscular activation to promote patient compliance with the rigid orthopedic brace.

14. A wearable orthopedic muscle stimulation system for treating an orthopedic condition affecting a musculoskeletal system of a patient, the system comprising:
a wearable garment configured to be worn by the patient in proximity to target musculature associated with the orthopedic condition;
an electrode array comprising a plurality of electrodes carried by the wearable garment;
a circuit device configured to generate and deliver at least one of transcutaneous electrical nerve stimulation (TENS) signals and electrical muscle stimulation (EMS) signals to the plurality of electrodes; and
a processor configured to:
determine, based at least in part on clinical imaging data of the patient, a stimulation map specifying which electrodes of the electrode array are to be activated and a stimulation modality to be delivered at each activated electrode based on a location of the activated electrode relative to the target musculature; and
control the circuit device to deliver electrical stimulation to the target musculature in accordance with the stimulation map.

15. The wearable orthopedic muscle stimulation system of claim 14, wherein the wearable garment comprises a compressive wrap portion having a double-layer configuration comprising an outer wrap layer and an inner elastic band, the inner elastic band configured to compress the electrode array against the patient to maintain electrode-to-skin contact across irregular body contours.

16. The wearable orthopedic muscle stimulation system of claim 14, wherein the electrode array comprises a plurality of individually addressable electrode elements, and wherein the circuit device is configured to selectively activate subsets of the individually addressable electrode elements based on the stimulation map without requiring physical repositioning of discrete electrode pads.

17. The wearable orthopedic muscle stimulation system of claim 14, wherein the plurality of electrodes comprise electrode pads adjustably positionable along an electrode positioning region of the wearable garment, the electrode positioning region configured to permit repositioning of the electrode pads to accommodate changes in the musculoskeletal system of the patient over the course of treatment.

18. The wearable orthopedic muscle stimulation system of claim 14, wherein the circuit device further comprises a wireless communication module configured to communicate with a companion application executing on a separate computing device, the companion application configured to receive session data from the circuit device and to transmit adjusted stimulation parameters to the circuit device.

19. The wearable orthopedic muscle stimulation system of claim 14, wherein the system further comprises an inertial measurement unit integrated into the wearable garment and communicatively coupled to the circuit device, the inertial measurement unit configured to detect an orientation and movement of the wearable garment, and wherein the processor is further configured to determine, based on data from the inertial measurement unit, whether the wearable garment is being worn by the patient during a given period.

20. A method for configuring a wearable orthopedic stimulation device for a patient presenting with scoliosis, the method comprising:
receiving diagnostic imaging of a spinal region of the patient;
assessing, from the diagnostic imaging, one or more Cobb angle measurements characterizing a degree and location of spinal curvature of the patient;
identifying, based on the one or more Cobb angle measurements, a concave aspect and a convex aspect of the spinal curvature;
determining a stimulation map for an electrode array of the wearable orthopedic stimulation device, the stimulation map specifying activation of a first subset of electrodes positioned in proximity to the convex aspect to deliver electrical muscle stimulation (EMS) and activation of a second subset of electrodes positioned in proximity to the concave aspect to deliver transcutaneous electrical nerve stimulation (TENS); and
transmitting the stimulation map to a circuit device of the wearable orthopedic stimulation device.
